(19) United States
(12) Patent Application Publication
(10) Pub. No.: US 2024/0123223 A1
(43) Pub. Date: Apr. 18, 2024

(54) TRIGGERING PERIPHERAL NERVE STIMULATION FOR RLS OR PLMD BASED ON SLEEP-RELATED DATA

(71) Applicants: Jonathan David Charlesworth, San Francisco, CA (US); Stephanie Kayla Rigot, Pleasanton, CA (US)

(72) Inventors: Jonathan David Charlesworth, San Francisco, CA (US); Stephanie Kayla Rigot, Pleasanton, CA (US)

(21) Appl. No.: 18/394,712

(22) Filed: Dec. 22, 2023

Related U.S. Application Data
(63) Continuation-in-part of application No. 17/987,471, filed on Nov. 15, 2022, now Pat. No. 11,878,166.
(60) Provisional application No. 63/279,774, filed on Nov. 16, 2021.

(51) Int. Cl. A61N 1/36

(52) U.S. Cl. CPC A61N 1/36003; A61N 1/36031; A61N 1/36034; A61N 1/06

(57) ABSTRACT

A technique for neurostimulation therapy is disclosed, particularly for patients suffering from Restless Legs Syndrome (RLS) or Periodic Limb Movement Disorder (PLMD). The technique can involve initiating a high-frequency electrostimulation signal during a specific time period associated with the patient's transition from sleep to wakefulness. The signal can be delivered to a target location on the patient at a frequency ranging from 500 Hz to 15,000 Hz and can be controlled toward parameters below the threshold that would wake the patient, while effectively mitigating the symptoms of RLS or PLMD.

### CLAIM OF PRIORITY

This application is a continuation-in-part of U.S. patent application Ser. No. 17/987,471, filed on Nov. 15, 2022, which claims priority to and the benefit of U.S. Provisional Application Ser. No. 63/279,774, filed on Nov. 16, 2021, each of which is hereby incorporated herein by reference, and the benefit of priority of each of which is claimed herein.

### TECHNICAL FIELD

This document pertains generally, but not by way of limitation, to neurostimulation devices, and more particularly to systems and methods for charging the devices for providing recurrent electrostimulation therapy sessions.

### BACKGROUND

Electrical nerve stimulation can be used to treat one or more conditions, such as chronic or acute pain, epilepsy, depression, bladder disorders, or inflammatory disorders. Certain neurological disorders can be attributed to overactivity of sensory or other peripheral nerve fibers which can disrupt quality of life, and/or the processing of such neural activity in the brain. Restless Legs Syndrome (RLS) and Periodic Limb/Leg Movement Disorder (PLMD) are two such neurological conditions that can significantly affect sleep in human patients. RLS (which can also be called Willis-Ekbom Disease (WED)) patients can experience uncomfortable tingling sensations in their lower limbs (legs) and, less frequently in the upper limbs (arms). RLS is characterized by an uncontrollable urge to move the affected limb(s). Such sensations can often be temporarily relieved by moving the limb voluntarily but doing so can interfere with the RLS patient's ability to fall asleep. PLMD patients can experience spontaneous movements of the lower legs during periods of sleep, which can cause the PLMD patient to wake up. RLS can be a debilitating sleep disorder and can be comorbid with other sleep disorders such as insomnia or sleep apnea syndrome (SAS).

### SUMMARY

The present inventors have recognized, among other things, a technique to help reduce the effects of restless leg syndrome or other sleep disorders. A wearable electrostimulation device can be applied a subject's leg at or near a nerve target. The wearable electrostimulation device can include or use auxiliary sensors to collect data corresponding such as to the subject's heart rate, oxygenation, or leg movement during a sleep session. Also, an auxiliary component such as a charger for the wearable device can be used such as to collect data corresponding to a sleep environment of the subject.

Collected data can be used such as to help a clinician prescribe treatments to sleep disorders, such as to help a clinician decide, e.g., when to apply the therapy, when to stop applying a therapy, or when to restart applying therapy. Several modalities of data collection can be included in a therapy system. This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

FIG. 1 depicts an example of a wearable external electrostimulation device shown located in proximity to a targeted region of a targeted peroneal nerve.

FIG. 2A depicts an example of a leg-wearable electrostimulation device in use on at a subject leg location.

FIG. 2B depicts an example of a leg-wearable electrostimulation device in use on at a subject leg location.

FIG. 3A depicts a pair of an example of leg-wearable electrostimulation devices wrapped around a local interface device.

FIG. 3B depicts front, side, and back views of an example of a leg-wearable electrostimulation device.

FIG. 4 schematically depicts an example of an electrostimulation system.

FIG. 5 depicts an example of several auxiliary sensors that can be included in an electrostimulation system.

FIG. 6 is a flowchart of a method of using an example of an electrostimulation system.

FIG. 7 shows an example of a closed-loop electrostimulation treatment system.

FIG. 8A shows an example of a technique to control RLS electrostimulation therapy delivery via the closed loop RLS electrostimulation therapy system.

FIG. 8B is a flowchart showing in establishing or adjusting at least one signal parameter based on feedback.

FIG. 9A depicts IMU data over time during a sensing period.

FIG. 9B depicts electromyography (EMG) data over time during a sensing period.

FIG. 10 is a flowchart illustrating a technique for administering neurostimulation therapy to a patient with Restless Legs Syndrome (RLS) or Periodic Limb Movement Disorder (PLMD).

FIG. 11 illustrates generally an example of a block diagram of a machine.

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

### DETAILED DESCRIPTION

The present techniques can help improve efficiency or effectiveness of treating a sleeping disorder such as RLS or PLMD, such as by issuing neural electrostimulation to a particular subject while using auxiliary components of an electrostimulation system to determine a quality of sleep of the subject during use of the electrostimulation system. In an example of sleep therapy such as electrostimulation therapy, a treatment routine or schedule can be prescribed for the subject by a sleep professional such as a clinician or sleep coach. A challenge of treating individuals with sleeping disorders is that few mechanisms exist for field research into the subject's native sleep routine. The sleep professional must rely on lab research of the subject, which can often be conducted in conditions which exacerbate causes of the sleep disorder, or the professional must rely on the subject's own account of sleep patterns and sleep environment. Further, it is especially difficult for the sleep professional or the subject to gain insight into symptoms exhibited by the subject during sleep in their native sleep environment, since the subject is unconscious, and the sleep professional is not present.

Subjects can vary in their response to medical treatments of sleep disorders. Thus, an approach to treatment using data-driven personalization of care can improve individual patient outcomes and to reduce individual or global treatment costs or treatment efforts. Compared to pharmaceutical therapies, electrical neurostimulation therapies have a particularly large potential for benefitting from personalization because such electrostimulation therapies are not necessarily monolithic. Instead, nerve stimulation can be optimized or adjusted, such as by programmatically adjusting one or more of the parameters of the electrical neurostimulation. The present inventors have conceived of a system for treating or monitoring sleep disorders by supplying electrostimulation to a subject along with detecting one or more sleep parameters associated to lack of sleep quality or a sleep disorder of the subject. The system can modify an electrostimulation protocol based on feedback associated with trends in sleep quality or sleep patterns detected by the system. The present inventors have also recognized, among other things, that a closed-loop or similar system that can adjust or optimize treatment quickly, e.g., using automation, thus improving individual patient treatment outcomes and reducing a cost of treatment.

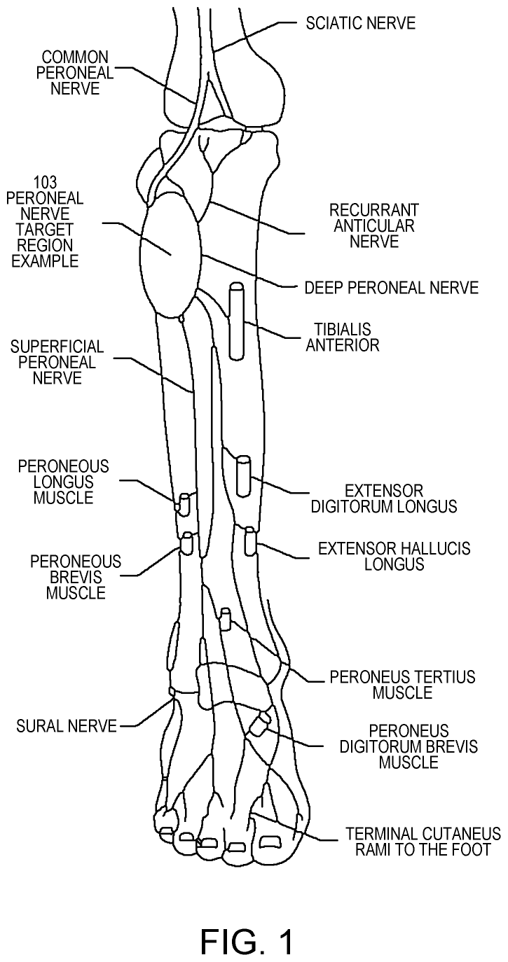

FIG. 1 depicts an example of a wearable external electrostimulation device shown located in close proximity to a targeted region 103 of a targeted peroneal nerve, such as at an anterior or lateral location just below the subject's knee, such as over a portion of the deep peroneal nerve. The wearable external electrostimulation device can be alternatively or additionally located at an even more inferiorly located region on the lower leg, such as over a portion of a superficial peroneal nerve, such as can innervate one or more parts of the tibialis anterior muscle. The superficial peroneal nerve and its direct peripherally extending branches and the sural nerve and its direct peripherally extending branches are the primary sensory nerves innervating this region of the leg. Therefore, such nerve locations are prime targets for RLS electrostimulation therapy. The present techniques can also include one or more specified selected nerve targets including such as a sural nerve, or a femoral nerve.

In an example depicted in FIG. 1 at least one electrode can be placed at the peroneal nerve target location, such as externally on the skin directly above or as close to possible to the superficial peroneal nerve. At least one electrode can be located directly below the bone landmark of the fibula in the outside of the knee below the lateral collateral ligament, or within 1 to 2 inches of the same. A second electrode may be located such that there is at least 1 inch of separation from the edge of the first electrode to the edge of the second electrode. The second electrode can be placed either along the length of the peroneal nerve, e.g., further down the leg, or the second electrode can be placed directly over the tibia, such as at about 1 to 2 inches below the first electrode. In an example, this second electrode can be located directly opposing the first electrode, to the inside of the knee, directly below the medial collateral ligament on the side of the tibia. The electrostimulation field may then be varied, such as to extend between a smaller or longer distance, such as to decrease sensory perception by the patient, if desired. In an example, two separate or different electrostimulation fields can be applied, such as by using the second electrode on the tibia as the common return electrode, such as can create a modulated electrostimulation field across the leg below the knee.

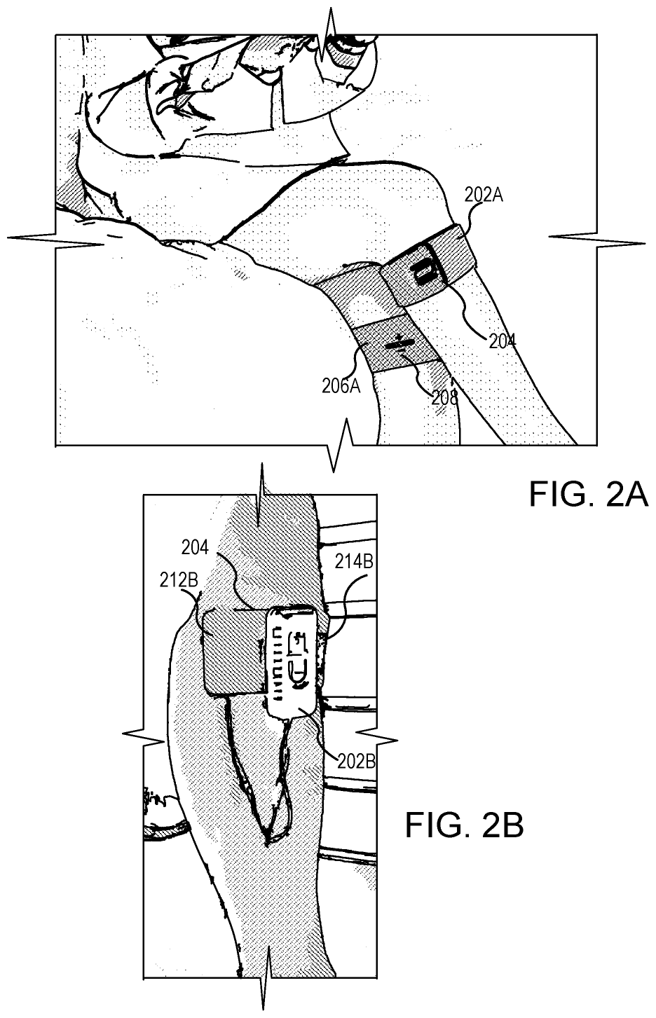

FIG. 2A and FIG. 2B depict an example of a leg-wearable electrostimulation device in use on at a subject leg location. For example, as depicted in FIG. 2A, a first leg-wearable electrostimulation device 202A and a second leg-wearable electrostimulation device 202B can be worn by the subject bilaterally on each leg at respective first and second leg locations. In an example, the leg-wearable electrostimulation device can obtain a two or more electrodes for delivering transcutaneous electrostimulation. As described above with respect to FIG. 1, the leg-wearable electrostimulation device can be attached or held to one of the first leg location 204 and second leg location 208, at a respective nerve target. While described herein as a leg-wearable electrostimulation device, the device can be sized and shaped to be able to be attached or held to one of several body locations of the subject, e.g., a leg, arm, foot, waist, neck, head, or chest of the subject. In an example depicted by FIG. 2A, the wearable electrostimulation devices 202A, and 206A can include or use a strap, sleeve, band, or clamp to help hold the electrodes to the skin of the subject. Also, as depicted in 2B, each wearable electrostimulation device can adhere to its corresponding leg location and be held thereto. Alternatively or additionally, as depicted in FIG. 2B, the wearable electrostimulation device can be sufficiently wearable on the skin surface of the patient by adhesion forces of the electrodes alone without the need for additional features to help hold the device to the subject.

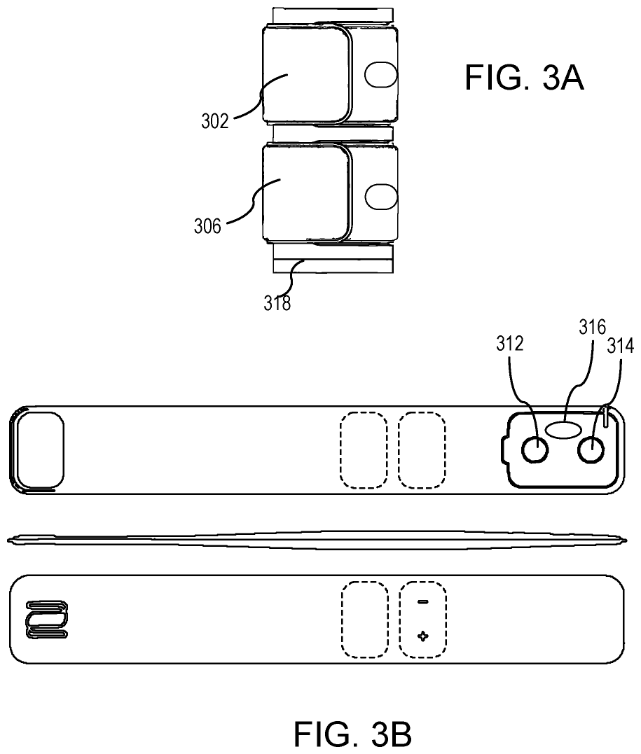

FIG. 3A depicts a pair of an example of leg-wearable electrostimulation devices 302 & 306 wrapped around a local interface device 318. The local interface device 318 a type docking station or charging station for each of the leg-wearable electrostimulation devices 302 & 306 and can be communicatively coupled thereto. The local interface device 318 can reside near a subject during sleep, such as on a nightstand or dresser or any other location near the sleep environment of the subject. The local interface device 318 can include or use at least one environment sensor for collecting environment data of a sleep environment of the subject. The environment sensor can collect data associated with luminosity, temperature, audio, or motion of a bedmate or other movement in the sleep environment of the subject. The local interface device 318 can include or use battery charging circuitry for charging a rechargeable battery of either of the first leg-wearable electrostimulation device 302 and the second leg-wearable electrostimulation device 306. The docking station charge the first and second leg-wearable electrostimulation devices 302 and 306 concurrently or simultaneously. The local interface device 318 can provide a safe or hygienic place to store the leg-wearable electrostimulation devices 302 and 306 such as to preserve a hydrogel material of the devices 302 and 306 or to minimize accumulation of dust and dirt near the electrodes. The local interface device 318 can include or use or be communicatively coupled to a processor or controller circuitry. The processor circuitry can receive a user input such as communications from a user interface (UI). The UI can include or use switches, buttons, knobs, touch panels, status LEDs, or display screens such as to enable user interaction for performing electrostimulation therapy. The display, status LEDs, or other similar components can be capable of displaying or indicating user data, test outcomes, or instructions. In one example the display can be an LCD screen embedded in either of the wearable electrostimulation. Alternatively or additionally, the user can interact with the leg-wearable electrostimulation device by means of the software application on a remote device such as a computer or a mobile phone.

FIG. 3B depicts front, side, and back views of an example of a leg-wearable electrostimulation device. The leg-wearable electrostimulation device can include or use a first skin electrode 312, a second skin electrode 314, and at least one auxiliary sensor 316. The auxiliary sensor can be located close to the first and second electrodes 312 & 314 such as to detect a condition at or near an electrostimulation site. Also, the auxiliary sensor can be located elsewhere on the band and relatively remote form the electrodes 312 & 314.

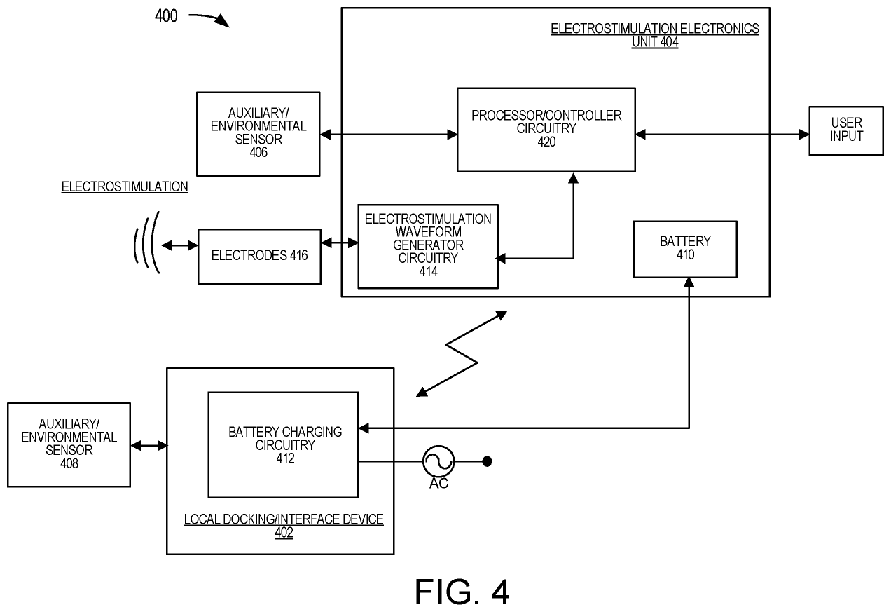

FIG. 4 depicts an example of an electrostimulation system. An electrostimulation system 400 can include or use an electrostimulation electronics unit 404 communicatively coupled to a local docking/interface device 402. The electrostimulation electronics unit 404 can include or use electrodes 416 for delivering a transcutaneous electrostimulation to a subject. The electrostimulation electronics unit 404 can be embedded within or coupled to a leg band, sleeve, or strap such as for holding the electrodes 416 to a treatment location. The electrostimulation electronics unit can also include or use electrostimulation waveform generator circuitry 414, a battery, and processor/controller circuitry 420. The local interface device 402 can be used local to the subject's sleep environment. The electrostimulation electronics unit 404 the local interface device 402 can include or use one or more auxiliary sensor 406 and one or more auxiliary sensor 408. As described further below, the auxiliary sensors described herein can be communicatively coupled to one or more processor circuit, such as the processor circuitry 420. In an example as depicted in FIG. 4, the local interface device 402 can be a charging station and can include or use battery charging circuitry 412. The battery charging circuitry 412 can be removably coupled to the battery 410 for a battery charging session. The local interface device 402 can also be a case, reservoir, cover, or other type of docking station for housing, maintaining, protecting, or routinely servicing the electrostimulation electronics unit.

### Examples of Sleep Environment Sensors and Other Auxiliary Sensors

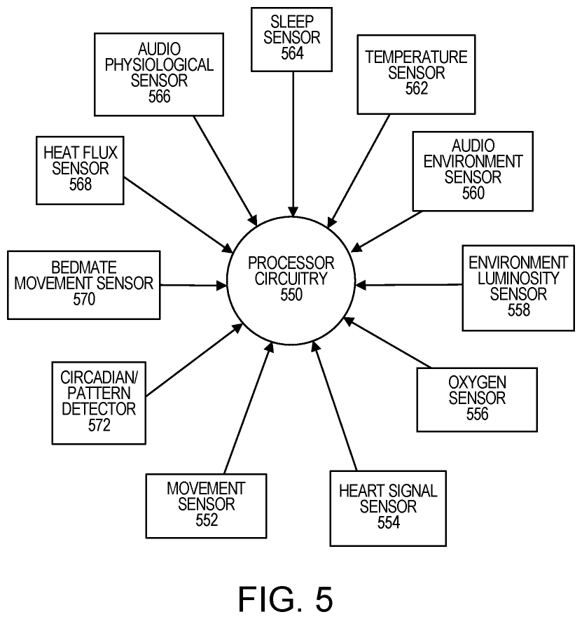

FIG. 5 depicts an example of several auxiliary sensors that can be included in an electrostimulation system. As explained herein, one or more environment sensors or other auxiliary sensors can be included on the leg band, adhesive patch, or other wearable carrier of the electrostimulation device located on one or both legs of the subject, or included at a local docking station, smartphone, or other local interface device in the sleep environment of the subject, or elsewhere in the sleep environment of the subject. Such sensors can be communicatively coupled to one or more processor circuit, such as for collecting the environment or other auxiliary data for monitoring, analyzing, or diagnosing and generating a response based on the auxiliary data. In general, while the device can include an impedance sensor, such as for sensing skin-electrode impedance, the environment or other auxiliary sensors described herein are configured to sense auxiliary data other than skin-electrode impedance, which can be useful for various other purposes, as explained herein. Some illustrative examples are described below.

1. A position, orientation, posture, or movement sensor can include an accelerometer, a gyro, a tilt switch, or other similar sensor such as to help enable collecting auxiliary data at a location at which the leg-wearable device is worn, such as on one leg of the subject, or with separate wearables that can be located bilaterally on opposing legs of the subject. Monitoring of position, orientation, posture or movement of the patient can enable signal processing, such as by a processor circuit, for determining one or more of duration, frequency, period, or amplitude of the movement. As an illustrative example, a position sensor can be used to determine a patient's leg movement or orientation or posture, e.g., one or more upright, recumbent, lateral decubitus, left lateral decubitus, right lateral decubitus, prone, supine, or the like. Such primary information can be analyzed by a processor for determining a response. Durations in such positions, transitions between such positions, frequency of transitions, or other secondary information derived from the position, orientation, or movement sensor can be determined and used by the processor for determining a response. To the extent that the position, orientation, posture, or movement sensor includes an accelerometer or microphone responsive to a tap or pattern of taps (e.g., from a fingertip of the subject) such a sensor can additionally or alternatively be used as a user-input device, such as for starting or pausing neurostimulation in response to a specified pattern of user taps from the subject.

2. A heart signal sensor can be located on the leg-worn wearable carrier (e.g., band or adhesive patch), such as to help enable collecting auxiliary data at a location at which the leg-wearable device is worn, such as on one leg of the subject, or with separate wearables that can be located bilaterally on opposing legs of the subject. The heart rate sensor can include an optical sensor, such as a photoplethysmography (PPG) sensor, or the heart signal can be derived from a blood pressure signal, such as can use a leg-worn band of the electrostimulation device carrier in a similar manner to a blood pressure cuff, from which a blood pressure signal can be derived, and a heart rate or heart morphology signal can be determined. The heart signal can additionally or alternatively be determined using an audio sensor, such as a microphone or accelerometer, which can be used to listen for heart sounds or blood pressure audio information. The heart sensor can include electrocardiogram (ECG) electrodes and sensing amplification circuitry that can be used to sense an electrical heart rate or heart morphology signal. The heart sensor can include an impedance sensor such as to sense a cardiac stroke signal by injecting a test current and measuring a response voltage—in this context, any skin-electrode impedance that is measured is simply present as a confounding component of the impedance signal from which the cardiac stroke component is to be extracted. The heart signal sensor can be used to determine one or more of heart rate, heart signal morphology, heart rate variability (HRV), or a secondary signal that can be derived from one or more of these. For example, a respiration signal can be derived from or correlated to HRV. A sleep or sleep-stage indication can also be derived from or correlated to HRV.

3. An oxygen sensor can be located on the leg-worn wearable carrier (e.g., band or adhesive patch), such as to help enable collecting auxiliary data at a location at which the leg-wearable device is worn, such as on one leg of the subject, or with separate wearables that can be located bilaterally on opposing legs of the subject. The oxygen sensor can include an optical sensor for detecting blood oxygenation auxiliary data (e.g., blood oxygenation saturation (SpO₂) auxiliary data) from the leg location at which the device is worn.

4. An environment luminosity sensor can be located on a docking station, smartphone, or other local interface device in a sleep environment of the subject, such as to help detect the ambient light level, which can affect sleep. Such information can help distinguish, for example, an arousal based on a light turning on from an arousal due to RLS or PLMD. The effect of a light being on, or sleep/wake behavior of turning a light on while lying awake can be monitored, analyzed, or both.

5. An audio environment sensor, such as a microphone, can be located on the wearable(s) (e.g., worn on one or both legs) or on a docking station, smartphone, or other local interface device in a sleep environment of the subject, such as to help detect the ambient sound or noise level, which can affect sleep. Such information can help distinguish, for example, an arousal based on a loud sound from an arousal due to RLS or PLMD. The effect of ambient sound (e.g., music), or sleep/wake behavior of turning music on while lying awake can be monitored, analyzed, or both.

6. A temperature sensor, such as a thermocouple or thermometer, can be located on the wearable(s) (e.g., worn on one or both legs) or on a docking station, smartphone, or other local interface device in a sleep environment of the subject, such as to help detect body temperature or ambient room temperature, both of which can affect sleep. The body temperature or ambient room temperature can be used with one or more other monitored sleep quality metrics, such as to control an environmental variable (e.g., room temperature) or therapy parameter (e.g., neurostimulation, drug titration, CPAP, or the like) to promote or optimize sleep quality.

7. A sleep sensor can be located on the wearable(s) (e.g., worn on one or both legs) such as to help detect or correlate one or more of sleep state, sleep onset, sleep termination, sleep stage, muscle atonia associated with sleep stage, body temperature associated with sleep stage, or the like. For example, the sleep sensor may combine information from one or more or a composite information from a heart sensor, a respiration sensor, or a patient activity sensor, such as to help determine the sleep-related information or indication of interest.

8. An audio physiological sensor can be located on the wearable(s) (e.g., worn on one or both legs) such as to help detect or correlate one or more of pulmonary, cardiopulmonary, blood pressure, respiration, or other information, such as can be obtainable via a microphone, accelerometer, or other audio sensor such as can be located on the wearable.

9. A heat flux sensor can be located on the wearable(s) (e.g., worn on one or both legs) such as to help detect or correlate one or more of peripheral circulatory functional information, such as vasodilation or vasoconstriction, such as may play a role in sleep quality and may be monitored or used for adjusting or recommending a therapy protocol (e.g., neurostimulation, CPAP, drug regimen, or other sleep-related therapy or protocol).

10. A bedmate movement sensor can be located on a docking station, smartphone, or other local interface device in a sleep environment of the subject, such as to help detect the movement of a bedmate (e.g., a person or pet sharing a bed or sleeping area with the subject, that is, some person or animal other than the subject), which can affect sleep. In an example in which both the subject and the bedmate are provided with wearable devices, the information about movement of the bedmate can be generated by a wearable worn by the bedmate (e.g., leg-worn, wrist-worn, or another device worn by the bedmate).

11. Circadian or other pattern detector can use information from one or more other physiologic auxiliary sensors, such as can be collected over a period of time and stored and analyzed for the presence of a circadian or other pattern that can be useful in recommending or adjusting a therapy or generating another response to such information.

In an example in which the wearables include a pair of wearable leg band or adhesive patch neurostimulation device carriers, data from the auxiliary sensors on each of the subject's left and right legs can be temporally synchronized such as for signal-processing analysis by the processor componentry, such as for providing one or more responses, such as described herein. Using bilateral auxiliary sensor data, as opposed to auxiliary sensor data from an auxiliary sensor on one leg, can allow correlation between auxiliary data on the different legs to filter out noise factors— such as the leg movements that are present in the RLS or PLMD patients wearing the wearable devices at the leg-worn locations of interest. This can obviate the need for a wrist-worn device, and appropriate noise-filtering of the leg movements, together with the bilateral auxiliary data stream, can help provide adequate auxiliary information relevant to sleep monitoring or one or more forms of sleep therapy.

### Examples of Responses to Environmental or Other Auxiliary Sensor Data

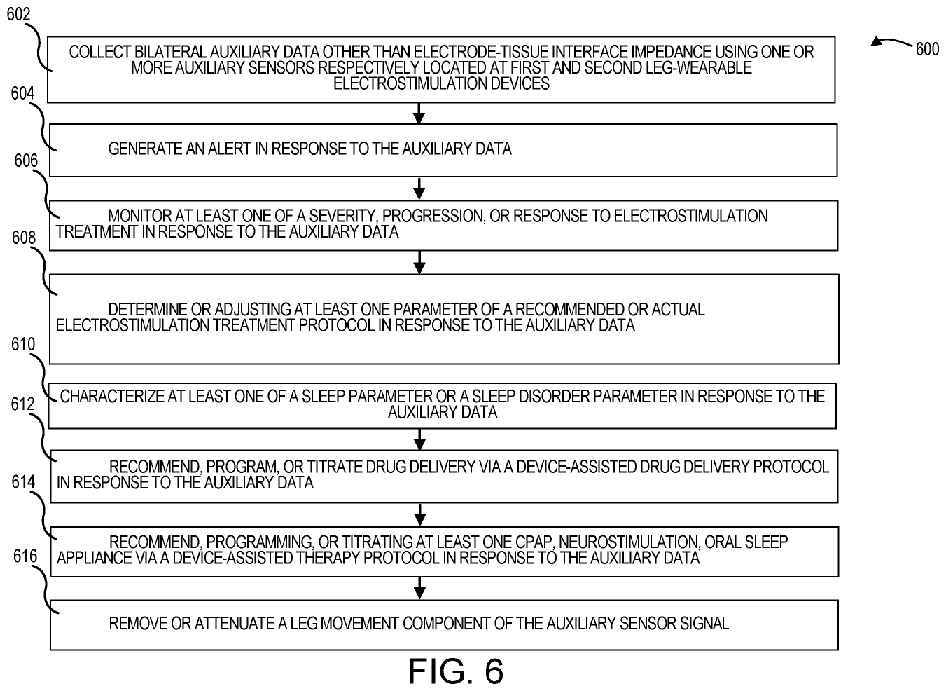

FIG. 6 is a flowchart of a method of using an example of an electrostimulation system. At 602, auxiliary data can be collected using one or more auxiliary sensors located respectively at one of the leg-wearable electrostimulation devices. The auxiliary data can be data other than electrode-tissue interface impedance. In an example, Bilateral auxiliary data can be collected using one or more auxiliary sensors respectively located at first and second leg-wearable electrostimulation devices. The auxiliary sensor data can be provided to a processor, such as for monitoring, analysis, diagnosis, signal-processing, storage, or generating a response. Illustrative non-limiting examples of responses can include:

At 604, Generating an alert to the subject or a caregiver, e.g., via a local or remote user interface device coupled to the processor, such as can be helpful toward the goal of improving sleep. The alert may be contemporaneous, or it may be delayed so as not to interfere with sleep by providing the alert.

At 606, Monitoring can include storing auxiliary data or information derived therefrom, such as for monitoring at least one of a severity or a progression of a sleep disorder or other sleep condition, or of an auxiliary physiologic response to electrostimulation treatment. In an example, the auxiliary physiologic response to electrostimulation constitutes something other than (or in addition to) an evoked response to neurostimulation or an EMG sensor response to neurostimulation from an auxiliary sensor located on the wearable.

At 608, Determining or adjusting at least one electrostimulation parameter of a recommended or actual electrostimulation treatment protocol (e.g., amplitude, frequency, duration, electrode selection, ramping, or any setting of one or more other neurostimulation parameter).

At 610, Characterizing at least one of a sleep parameter or a sleep disorder parameter. This can include, for example, characterizing one or more of sleep state, sleep stage, sleep quality, OSA degree, CSA degree, OSA vs. CSA, arousal frequency, arousal duration, physiological or environmental factors correlated to the sleep or arousal parameter, or the like.

At 612, Recommending, programming, or titrating drug delivery via a device-assisted drug delivery protocol. This can include, for example, dosage amount, dosing frequency, dose ramp-up or ramp-down, logging a history of environment or other auxiliary parameters or correlation with drug delivery, or the like, such as via a smartphone application or other user-interface device for the subject or a caregiver.

At 614, Recommending, programming or titrating at least one of CPAP, neurostimulation, oral sleep appliance via a device-assisted therapy protocol. This can include, for example, for one or more such non-drug therapy, non-drug therapy amount, therapy frequency, therapy ramp-up or ramp-down, logging a history of environment or other auxiliary parameters or correlation with non-drug therapy delivery, or the like, such as via a smartphone application or other user-interface device for the subject or a caregiver.

And, at 616, Removing or attenuating a leg movement component of the auxiliary sensor signal. As described further below, electrostimulation systems described herein can include or use various mechanisms such as to mitigate noise from the data. For example, a subject having one or more symptoms of RLS can produce leg movement which can be challenging to interpret from data collected by one or more movement sensors. Other sensor-inputs can be used such as to attenuate this signal or remove this leg movement component from an analysis of data collected by the auxiliary sensors.

### Use of Additional Sensors to Measure Objective Metrics Relating to Sleep

1. Activity and motion sensing. The system can include or use additional sensors such as an accelerometer, a gyroscope or inertial measurement unit (IMU) to measure activity. While traditional actigraphic sensors record absence of motion to impute sleep states and stage them, patients suffering from Restless Legs Syndrome (RLS) may have unique physiological differences that makes it difficult to do the same without accounting for natural periodic leg movements (PLMs) or other voluntary leg movements (such as rubbing feet together, kicking legs, stretching, dorsiflexion) that RLS patients often repeat constantly through the night, sometimes even when in light sleep. Such movements may falsely register as wakefulness when they are not accounted for. Further to this, the system can allow for a treating physician to only see movement that is relevant to arousals from sleep, as opposed to movements that may have been involuntary or otherwise unimpactful to sleep quality. This may be derived from a combination of processed data from both legs along with movement data from the bedside dock. As an example, it may be the case that only side-to-side turns during sleep cause an actual arousal (as measured by surface EEG) and other leg movements may be unimpactful to sleep. The on-board gyroscope allows for detection of rotational movement and distinguishes these from leg movements detected by the accelerometer.

2. Heart Rate and sympathetic tone. The system could include measurement of average heart rate using a PPG sensor, or by directly coupling ECG signal from the hydrogel stimulation electrodes. Measurement of heart rate, or ECG is often complicated by movement artifacts and noise and in the case of PPG sensors, skin tone as well. Our system presents a unique advantage in that the bedside base station can process data from the bilateral devices worn on both legs to time synchronize, average, and obtain a more reliable background heart rate even when individual sensor data may appear to be noisy. The use of the bedside dock further allows for motion cancelation (by sensing patient movement using a camera, infrared or electromagnetic signal emitted).

3. Spo2/Oxygenation. The system could include the measurement of oxygen saturation in the blood, an important marker relating to severity of conditions such as obstructive sleep apnea but also a potential key marker for poor circulation in the periphery. The system allows for synchronizing dynamic data from two legs at the same time via communication with the bedside base station and would employ the same motion canceling algorithms that benefit ECG and HR measurement described above in (2). The proposed skin surface over which the device is worn on the legs is rich with blood supply and serves as a prime target to measure oxygen saturation. Patients with RLS may also present with undiagnosed or otherwise poorly controlled sleep apnea, and the data presented to the physician would allow for accurately identifying these conditions that may be severely detrimental to the patient's health in the long-term.

4. Audio-based sensors. Use of an on-board microphone allows for detection of snoring or other apnea-related sounds. The microphone may be placed on the bedside charging dock and the use of this additional channel of data could further help refine sleep staging. The use of a microphone also allows the system to account for any ambient or background noises and their decibel level to correlate to sleep quality impacts.

5. Light sensors. Use of a photodetector or light sensor allows for the system to accurately tell when the patient attempts to fall asleep, an important metric to assess sleep onset latency. Most other methods rely purely on subjective patient reports to record when a patient went to bed. The use of an on-board light sensor either on the bands or the dock (or both) allows for the use of a combination of light, sound, and motion to accurately make a determination of time at which patient goes to bed without relying on subjective patient reports.

6. Environment sensors. Use of a thermistor (temperature sensor) and humidity sensor allows the system to further record environmental conditions around the use of the devices. This closely ties in to interpreting metrics that may negatively or positively impact sleep quality and can be used to coach and improve sleep hygiene and therefore sleep quality.

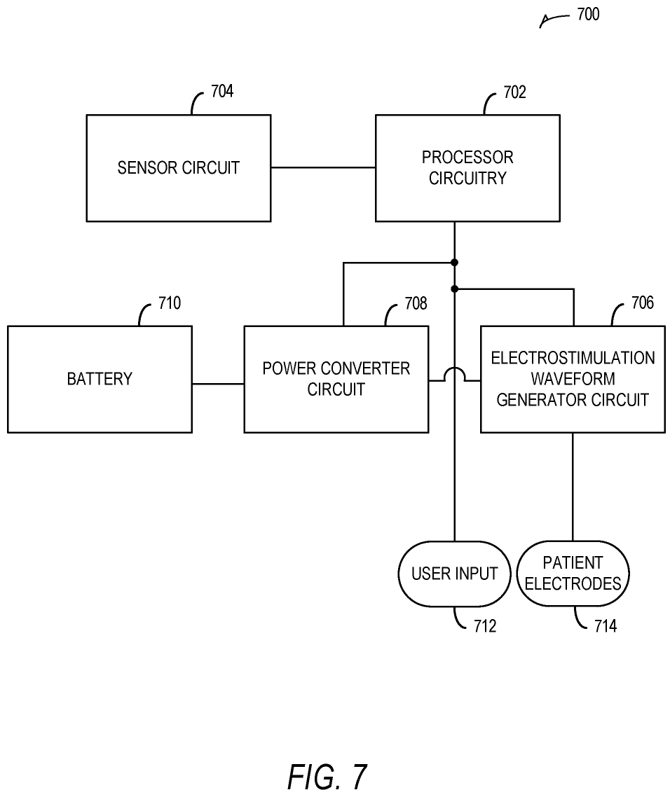

FIG. 7 shows an example of portions of a closed-loop electrostimulation treatment system 700. One approach to electrostimulation to provide therapy for Restless Legs Syndrome (RLS) or Periodic Limb Movement Disorder (PLMD) involves “user-triggered” electrostimulation. In user-triggered electrostimulation, delivery of a HF electrostimulation signal, via an electrostimulation device to mitigate a symptom of RLS or PLMD, can be commenced or terminated via manual activation/deactivation by the patient (e.g., commenced after the patient has experienced the symptom). In certain situations, such as after an onset of sleep of the patient, certain user-triggered electrostimulation approaches can be challenging. For example, during a period of the patient's sleep in which the electrostimulation device is not providing the HF electrostimulation signal (e.g., when the device is powered-down or in a reduced-functionality power-saving mode), symptoms from the patient can escalate until the patient experiences significant enough sleep arousal to awaken enough to manually initiate electrostimulation therapy via the electrostimulation device. For example, an exemplary user-triggered, open-loop session approach can involve, on average, ˜9 minutes from a patient waking before the patient manually initiates delivering of the HF electrostimulation signal, can additionally involve an average of −16 minutes from the manual initiation before the patient falls back asleep. The present inventors have recognized a need for a technique for automatic triggering of electrostimulation during patient sleep. This can help the patient stay asleep after sleep onset while mitigating one or more RLS or PLMD symptoms that may arise during sleep. For example, automatic initiation of delivery of the HF electrostimulation signal can be triggered based on received sensor data during a present sleep session (e.g., concurrent sensor data or sensor data collected within a specified period of time, such as the last ˜60 minutes). Alternatively or additionally, automatic initiation of delivery of the HF electrostimulation signal can be triggered based on historical sensor data or other data corresponding with at least one prior sleep session of the same patient (e.g., sensor data or other data corresponding with a sleep session greater than a specified period of time such as ˜24 hours before the present sleep session). Following initiating delivering the HF electrostimulation signal, the HF electrostimulation signal can be controlled such as to mitigate at least one target RLS or PLMD symptom of the patient while remaining subthreshold to the patient waking. Such control of the HF electrostimulation signal (e.g., establishing or adjusting at least one signal parameter of the HF electrostimulation signal) can be based on sensor or other data corresponding with the present sleep session or prior sleep sessions. A closed loop electrostimulation approach, such as described herein, can be particularly advantageous for certain RLS patients attempting to reduce or downtitrate or otherwise augment pharmaceutical drug therapy for RLS or PLMD and replace at least a portion of the drug therapy with electrostimulation therapy and to treat at least one target RLS or PLMD symptom with a similar efficacy. For example, closed loop electrostimulation can more-closely resemble (and therefore more effectively replace) a symptom relief efficacy and a patient self-administration involvement of the drug therapy, as compared to certain user-triggered electrostimulation approaches.

In FIG. 7, the RLS treatment system 700 can include processor circuitry 702, a sensor circuit 704, a battery 710, a power converter circuit 708, an electrostimulation waveform generator circuit 706, a user input device 712, and patient electrodes 714. The patient electrodes can include external electrodes, such as can be located on an adhesive skin-patch, such as for transcutaneous application of electrostimulation energy. For example, the patient electrodes can be positioned at or near a targeted nerve (e.g., a sural nerve, a peroneal nerve, a tibial nerve, etc.). The processor circuitry 702 can include a microprocessor, microcontroller, programmable logic circuit, or the like, such as can be powered by the battery 710 or other power source. The battery can be coupled to a power converter circuit 708, such as can include one or more of a buck power converter circuit, a boost power converter circuit, a buck-boost power converter circuit, or other inductive or capacitive or other circuit for converting the battery voltage and current to a desired output voltage and current such as for delivering electrostimulation to the subject via the patient electrodes 714. An electrostimulation waveform generator circuit 706 can receive a converted power signal from the power converter circuit 708, and can generate a suitable electrostimulation waveform, such as a HF electrostimulation waveform such as described herein. For example, the electrostimulation waveform generator can be configured by the processor circuitry 702 to generate a HF electrostimulation controlled-current waveform such as having a frequency within a range of about 500 Hertz (Hz) and about 15,000 Hz, or within a range of about 4000 Hz to about 5000 Hz. The electrostimulation waveform generator can also be configured to generate the HF electrostimulation controlled-current waveform including a current amplitude, e.g., controlled by the processor circuitry 702, within a range of 5 milliamperes to 30 milliamperes (e.g., at a level of 5 mA, 10 mA, 15 mA, 20 mA, 25 mA, or 30 mA, or finer resolution if desired). A similar electrostimulation device, including an electrostimulation waveform generator, is described in US Patent Publication Serial No. 2019/0083784 which is incorporated by reference herein in its entirety, including for its teaching of an electrostimulation device and techniques for waveform generation to treat at least one symptom of RLS or PLMD.

The sensor circuit 704 can include or be communicatively coupled to at least one sensor (e.g., auxiliary sensor 316 as depicted in FIG. 3A) to transmit sensor data to the processor circuitry 702. For example, the at least one sensor can include the movement sensor 552, heart signal sensor 554, oxygen sensor 556, environment luminosity sensor 558, or audio environment sensor 560 temperature sensor 562, sleep sensor 564, audio physiological sensor 566, heat flux sensor 568, bedmate movement sensor 570, circadian pattern detector 572, or other sensor such as described above with respect to FIG. 5. Generally, the sensor data received by the processor circuitry 702 via the sensor circuit 704 can be processed such as for determining a sleep arousal time period associated with actual or predicted transition from asleep to awake, or processed for controlling the HF electrostimulation signal to remain subthreshold to the patient waking while maintaining the delivery of the HF electrostimulation signal for mitigating a target RLS or PLMD symptom. The sensor data, received via the sensor circuit 704, can also be used to determine or receive an indication of sleep onset of the patient, e.g., to trigger downstream sensing or data processing for initiating delivery or control of the HF electrostimulation signal.

For example, the sensor circuit 704 can include or be communicatively coupled to an inertial measurement unit (IMU) for transmitting motion data of the patient during wearing of the patient electrodes 714. The IMU can include an accelerometer, a gyroscope, a magnetometer, or a combination thereof. The IMU can transmit linear acceleration data or angular velocity data, such as for a plurality of axis (e.g., an x-axis, a y-axis, and a z-axis). The IMU can be worn by the patient, e.g., can be located at or near a leg-wearable electrostimulation device 202 such as depicted in FIG. 2A. The motion data received from the IMU can be used by the processor circuitry 702, such as to determine past or present movement or predict future movement of a patient limb, e.g., at or near a patient nerve target. The motion data can be used by the processor circuitry 702 to control at least one parameter of the HF electrostimulation signal. For example, delivery of a HF electrostimulation signal can be triggered or initiated upon receiving motion data indicating a specified number (e.g., greater than about 5 or about 10) of leg movements within a specified first sensing duration (e.g., within a duration that can be specified at 1 minute, 2 minutes, 5 minutes, or the like), is received from the IMU. For example, the leg movements can be periodic leg movements, movements/positional information that is correlative with patient-subjective RLS symptoms during wake, or movements/positional information that is correlative with a likelihood of a patient waking. This can be useful, for example, in a PLMD patient who can experience leg twitches or motions while sleeping, such as to automatically turn on (or increase) therapy to help mitigate symptoms to help the patient stay asleep. Also, at least one parameter of the HF electrostimulation signal (e.g., a frequency, amplitude, pulse-width, etc.) can be established or adjusted based on motion data, corresponding with leg twitches or motions, within a specified second sensing duration, such as received via the IMU. For example, the second sensing duration can be concurrent with the HF electrostimulation signal being delivered to the patient (e.g., persist beyond a duration that can be specified at 5 minutes, 10 minutes, or the like). In an example, electrostimulation can be turned off (or can be ramped down or ramped off) where motion data is received indicating fewer than a specified number (e.g., less than about 10, about 5, or about 2) of leg twitches or motions or motion data not indicative of leg twitches or motions, the motion data received over a third sensing duration (e.g., within a duration that can be specified at 1 minute, 2 minutes, 5 minutes, or the like).

In an example, the sensor circuit 704 can include an impedance measurement circuit for transmitting a load impedance signal or other impedance data to the processor circuitry 702. For example, the load impedance signal can be generated by the impedance measurement circuit by issuing a known or baseline voltage amplitude signal and measuring a response current signal (or vice-versa), such as to calculate an approximated impedance. In an example, the response signal data or calculated impedance data can be logged, such as by storing it to a memory location, such as can be included in or coupled to the processor circuitry 702. In an example, the load impedance signal can be used as an indication of whether the electrodes 714 are in good condition, being worn (or properly worn) by the patient. For example, the impedance measurement circuit can detect reduction in impedance below a specified threshold, thereby infer that the patient has placed the device on their leg, and thus begin triggering downstream sensing or data processing for initiating delivery or control of the HF electrostimulation signal such as to help alleviate RLS or PLMD symptoms. In another example, the impedance measurement circuit can detect increase in impedance above a specified threshold, infer that the patient has removed the device from their leg, and terminate this process. In an example, the impedance measurement circuit or the processor circuitry 702 can determine whether the load impedance signal is within a specified nominal range indicating the electrodes 714 are in good condition and being properly worn by the patient. Here, the impedance measurement circuit or processor circuitry 702 can include or use one or more comparator circuits, which can be provided one or more reference values for comparison for establishing the nominal impedance range. If it is determined that the measured impedance data is outside the nominal range and the electrodes 714 are not being worn by the patient, the processor circuitry 702 can enter a power-saving mode and terminate or interrupt a downstream sensing period. Similarly, sensor data from any of the temperature sensor 562, sleep sensor 564, audio physiological sensor 566, heat flux sensor 568, bedmate movement sensor 570, circadian pattern detector 572 (as described with respect to FIG. 5) or a dedicated capacitive sensing circuit can be used to determine either that a leg-wearable electrostimulation device 202 (as depicted in FIG. 2A) is not being worn or that sleep onset of the patient has not commenced or is not imminent. For example, data received via sensors 562-572 can be used to infer or predict sleep onset, such as by monitoring a sleep environment (e.g., via audio physiological sensor, bedmate movement sensor 570, or circadian pattern detector 572). Also, data received via sensors 562-572 can be used to calculate or predict sleep onset by patient physiological information (e.g., via temperature sensor 562, sleep sensor 564, or heat flux sensor 568). Here, processor circuitry 702 can enter the reduced power mode and terminate or interrupt the downstream sensing period. Other device data, such as electrostimulation device charging data, a user input from the patient received via the user input device 712, or data indicated a present time of day or time of previous user activated electrostimulation can be similarly used by the RLS treatment system to infer, determine, or predict either that a leg-wearable electrostimulation device 202 (as depicted in FIG. 2A) is not presently being worn, or that mid-night awakenings are not imminent, or that sleep onset of the patient has not commenced or is not imminent, and the processor circuitry 702 can thereby enter the reduced power mode and terminate or interrupt the downstream sensing period. For example, the processor circuitry 702 can infer that sleep onset of the patient has not commenced or is not imminent based on charging data indicating the device is in a docking station (e.g., not being worn by the patient). As such, the processor circuitry 702 can facilitate a tiered approach to patient sensing, such as for eventually determining a sleep arousal time period associated with actual or predicted transition from asleep to awake: i) a standby period during which the processor circuitry 702 is not actively determining a sleep arousal time period and remains waiting (e.g., in the reduced power mode) for a trigger to commence a present sensing period, and ii) a present sensing period during which a sleep arousal time period is determined.

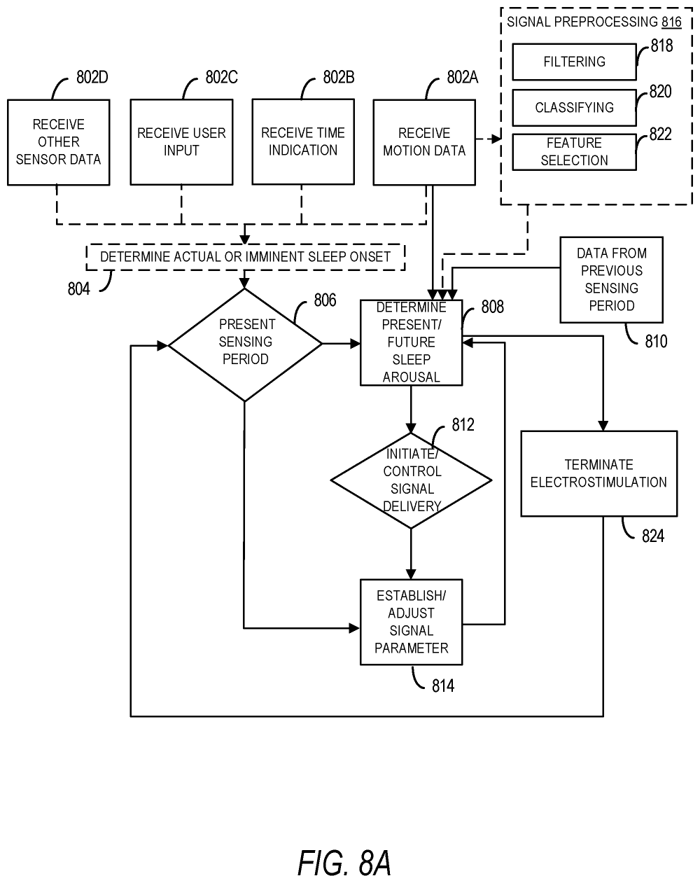

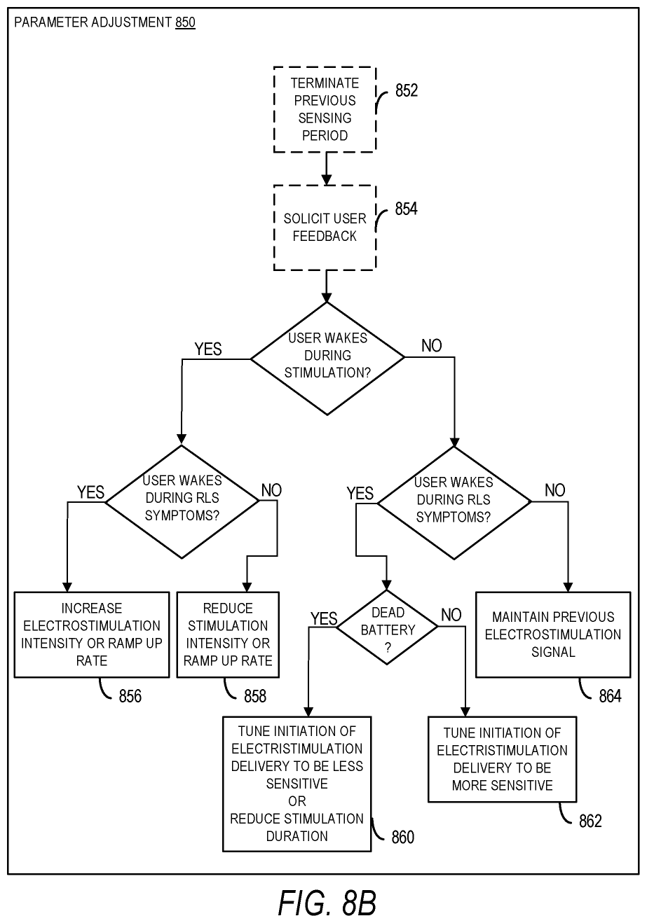

FIG. 8A shows an example of portions of a technique 800 for using the processor circuitry 702, using sensor data from the sensor circuit 704, the user input device 712, or both to control RLS electrostimulation therapy delivery via the closed loop RLS electrostimulation therapy system 700. At 802, the processor circuitry 702 can receive at least one of motion and/or position data from a motion and/or position sensor such as the inertial monitoring unit (IMU) (802A), a time indication (802B), a user input (802C), or other sensor data. The processor circuitry 702 can use such received information in determining whether to commence a present sensing period 806. Optionally, at 804, the processor circuitry 702 can use the at least one input 802A-802D to help determine whether the patient is attempting to fall asleep, whether onset of sleep has been detected, or whether onset of sleep is imminent (e.g., predicted sleep onset within a specified time period, such as within the next ˜30 minutes).

For example, at 804 the processor circuitry 702 can determine whether the patient is attempting to fall asleep or whether onset of sleep is imminent, such as based on the received time indication 802B, such as an indication of a present time of day or an actual or average time of day of one or more previous user-activated electrostimulation deliveries (e.g., from one or more respective previous sensing periods). For example, the patient can actuate a switch or can provide other user input at 802C, such as for signaling to the system 700 that the patient is intending to fall asleep. In an example, received motion data 802A from the IMU can be used to determine a position of the patient (e.g., upright vs. recumbent) or of the patient's lower limb, or whether leg activity movement indicates one or more RLS symptoms, such as leg twitches or motion, or is indicative of a patient attempting to sleep or of sleep onset. For example, the processor circuitry 702 can analyze the received motion data 802A from the IMU for a motion signature suggestive of a patient beginning wearing a leg-wearable electrostimulation device or a motion signature suggestive of a patient getting into bed. For example, the motion signature may begin with a large amplitude motion indicating that the patient is moving from a standing or seated position to a more horizontal position. As the patient approaches a reclining, supine or even prone position, the amplitude of the motions gradually decrease until they become imperceptible. In an example, the received other sensor data 802D can include heart rate (HR) data (e.g., via the patient electrodes 714 or via separate electrodes that can be placed or located in contact with the patient), can be used by the processor circuitry 702 to calculate a heart rate variability (HRV) parameter from the sensed heart rate signal. Here, HRV can be used to detect sleep or to detect a particular state of sleep. Also, a the received other sensor data 802D can include a respiration (breathing) signal (e.g., via the patient electrodes 714 or via separate electrodes that can be placed or located in contact with the patient), e.g., determined using an impedance sensor to detect respiration. Sleep state information can additionally or alternatively be extracted from the respiration signal, such as by signal processing such as can be performed by the processor circuitry 702. The received other sensor data 802D can also include sleep state information, e.g., obtained by interfacing with another sleep monitoring product that a patient may use, such as can communicate this information to the processor circuitry 702.

Alternatively, an actual or imminent sleep onset of the patient need not be determined by the processor circuitry 702 and rather can be expected or assumed, e.g., based on an indication of a related event received via inputs 802A-802C. For example, the related event can include a change in charging status of a leg-wearable electrostimulation device (e.g., an indication the device has been removed or decoupled from a device charger), an indication that the leg-wearable electrostimulation device has been placed on or near a patient limb.

Regardless of whether, at 804, actual/imminent sleep onset is determined by the processor circuitry 702, the processor circuitry 702 can commence a present sensing period at 806. For example, a present sensing period can involve increased processing and data analysis as compared to a reduced power mode (e.g., before commencing the present sensing period). At 808, during the present sensing period 806, the processor circuitry 702 can determine an actual or predicted sleep arousal, hypnopompia, or patient transition from asleep to wake. For example, the processor circuitry 702 can determine a sleep arousal time period during which the actual sleep arousal occurs or the predicted sleep arousal is predicted to occur. The sleep arousal time period can be relatively short and specific (e.g., within a period less than about 15 minutes, less than about 5 minutes, or less than about 2 minutes). Such a determination of the sleep arousal time period can be based on received motion data 802A during the present sensing period, received data associated with at least one previous sensing period 810, or both. For example, received motion data 802A from the IMU can be used to determine a position of the patient (e.g., upright vs. recumbent) or of the patient's lower limb, or whether leg activity movement indicates one or more RLS symptoms, such as leg twitches or motion. Such determination of a position of the patient can be used to determine the actual or predicted sleep arousal, hypnopompia, or patient transition from sleep to wake. Also, the processor circuitry 702 can analyze the received motion data 802A from the IMU for a motion signature suggestive of actual or predicted sleep arousal. For example, the motion signature can include a rapid, repetitive increase in motion or motion associated with a particular limb of the patient or with a particular region of the patient's body (e.g., levodopa associated with foot motion of a patient having RLS). Further, the processor circuitry 702 can evaluate whether the motion data 802A includes recorded motion during the anticipated sleep session from the previous night that is indicative of an RLS condition (e.g., based on a clinical indication of RLS symptoms or user feedback of diagnosed RLS symptoms from the previous night). In an example, the received other sensor data 802D can include heart rate (HR) data (e.g., via the patient electrodes 714 or via separate electrodes that can be placed or located in contact with the patient), can be used by the processor circuitry 702 to calculate a heart rate variability (HRV) parameter from the sensed heart rate signal. Here, HRV can be used to detect or predict sleep arousal. Also, a the received other sensor data 802D can include a respiration (breathing) signal (e.g., via the patient electrodes 714 or via separate electrodes that can be placed or located in contact with the patient), e.g., determined using an impedance sensor to detect respiration. Sleep state information can additionally or alternatively be extracted from the respiration signal, such as by signal processing such as can be performed by the processor circuitry 702. The received other sensor data 802D can also include sleep state information, e.g., obtained by interfacing with another sleep monitoring product that a patient may use, such as can communicate this information to the processor circuitry 702.

Where the determination of the sleep arousal time period is based on received motion data 802A during the present sensing period, the processor circuitry 702 can make the determination based on motion data received within a specified past time frame, e.g., the past hour. Here, the sleep arousal time period can be associated with an actual transition from asleep to awake based on present motion data, such as a change in orientation or motions or twitches above a specified threshold the patient is expected to wake. Alternatively or additionally, the sleep arousal time period can be associated with a predicted transition from asleep to wake, e.g., based on motion data indicating RLS motions or twitches from the specified past time frame, or otherwise indicating the patient will soon wake or experience sleep arousal. Where the determination of sleep arousal time period is based on received data associated with at least one previous sensing period 810, the processor circuitry 702 can perform data analysis of historical data to help forecast the sleep arousal time period during the present sensing period. In an example, the historical data corresponds with a single, same patient as that of the present sensing period and can be associated with one or more previous sensing periods of the same patient. For example, the historical data can include an average time during which a user typically activates open-loop electrostimulation signal, an average time during which a user typically experiences leg movements or other RLS or PLMD symptoms (e.g., based on motion data from one or more previous sensing periods), or user feedback associated with an individual previous sensing period. Here, the historical data can include waveform data corresponding with an electrostimulation administration during the one or more previous sensing periods and patient feedback data (e.g., user input data, motion data, or other sensor data) detected during the one or more previous sensing periods and corresponding with the waveform data. In another example, the historical data corresponds with a plurality of different RLS or PLMD patients (e.g., from one or more respective previous sensing periods) and can indicate one or more trends for an average time an RLS or PLMD patient will typically experience leg movements or other RLS or PLMD symptoms.

At 812, upon determining actual or predicted sleep arousal at 808, the processor circuitry 702 can initiate delivery of a HF electrostimulation signal (e.g., via the electrostimulation waveform generator circuit 706 of FIG. 7) or control the HF electrostimulation signal to remain subthreshold to the patient waking while maintaining a delivery of the HF electrostimulation signal to mitigate a target RLS or PLMD symptom. In one example, for therapy delivery upon detection of recent waking, “subthreshold” would refer to stimulation that does not interfere with the patient's ability to re-initiate sleep or does not reduce the likelihood of re-initiating sleep. In another example, for therapy delivery upon detection of imminent waking, “subthreshold” would refer to stimulation that does not interrupt sleep and does not increase the likelihood of waking. Herein, “subthreshold” to the patient waking up can refer to an electrostimulation waveform that does not induce phasic muscle contractions, does not induce changes in perception on a timescale of <10 seconds, and that does not induce sharp sensations of electrostimulation (like other low frequency (LF), e.g., <150 Hz transcutaneous electrical neurostimulation (TENS) waveforms). In an example, similar to that described with respect to FIG. 2A, a RLS treatment system 700 can include at least two leg-wearable electrostimulation devices for delivering bilateral electrostimulation to two or more limbs of the patient. Here, the processor circuitry 702 can initiate delivery of the HF electrostimulation signal (e.g., concurrent delivery commenced at approximately equal times) to both a first and a second leg-wearable electrostimulation devices based on movement detected only via the first leg-wearable electrostimulation device.

At 814, the processor circuitry 702 can establish or adjust at least one signal parameter during the present sensing period and based on one of the inputs 802A-802D, the received data associated with at least one previous sensing period 810, or the determination of actual or predicted sleep arousal. For example, the processor circuitry 702 can initially establish at least one signal parameter (e.g., an amplitude, a frequency, a pulse-width, intensity ramping rate etc.) at or near the commencing the present sensing period. For example, the initially established at least one signal parameter can be a default parameter or a prescribed starting parameter, e.g., based on at least one received data associated with at least one previous sensing period 810. Concurrent with the delivering the HF electrostimulation signal, the processor circuitry 702 can adjust the at least one signal parameter based on feedback, such as based on a determination of actual or predicted sleep arousal 808.

At 824, the processor circuitry 702 can terminate electrostimulation based on a determination that the actual or predicted sleep arousal, hypnopompia, or patient transition from asleep to wake has ceased or otherwise been effectively mitigated or prevented. For example, the processor circuitry 702 can terminate electrostimulation based on leg movements (e.g., above a specified intensity or value) ceasing for a specified period of time (e.g., for about two minutes, about five minutes, or about 10 minutes). In an example, other sensor data from any of the temperature sensor 562, sleep sensor 564, audio physiological sensor 566, heat flux sensor 568, bedmate movement sensor 570, circadian pattern detector 572 (as described with respect to FIG. 5), or HRV data/respiration data can be used to determine sleep arousal has ceased or otherwise been effectively mitigated or prevented. Terminating electrostimulation upon mitigation or prevention of the actual predicted sleep arousal can help prolong battery life and prevent unnecessary disturbance to sleep. In an example, following a termination of the electrostimulation signal at 824, the processor circuitry 702 can revert to the beginning of commencing the present sensing period or instead commence a new, subsequent sensing period similar to the present sensing period.

In an example, the processor circuitry 702 can establish or adjust at least one signal parameter 814 according to the method described in the flowchart of FIG. 8B. The parameter adjustment 850 can involve incorporating feedback (e.g., motion data, user input data, or other sensor data from the present sensing period or a past sensing period) or other input (e.g., data from sensors 562-572, an HRV or respiration sensor, etc.) to help determine whether the patient wakes during electrostimulation and whether the patient wakes during experiencing RLS symptoms. Based on the feedback or other input, the parameter adjustment 850 can involve adjusting the HF electrostimulation waveform and thereby help control the waveform toward parameters that can be established such that the HF electrostimulation is subthreshold to the patient waking while promoting the mitigation of at least one target RLS or PLMD symptom. In an example, the feedback or other input can include a user input based on a previous sensing period. For example, optionally at 852, the parameter adjustment 850 can involve terminating a sensing period (e.g., upon the patient waking up or another indication a patient no longer wishes to fall asleep). Upon termination of the sensing period, optionally at 854 the parameter adjustment 850 can involve soliciting user input (e.g., via the user input device 712 of FIG. 7). Here, the user input can include answers the questions of whether the patient awoke during stimulation and whether the patient awoke during experiencing RLS or PLMD symptoms. With respect to the parameter adjustment 850, a determination that the patient “awoke” can be equivalent to determining the patient experienced sleep arousal or entered hypnopompia. In another example, the feedback or other input can be based on present received motion data 802A (as depicted in FIG. 8A) or data associated with at least one previous sensing period 810. Here, the parameter adjustment 850 can involve analyzing the received data 802A or 810 to determine whether the patient awoke during stimulation and whether the patient awoke during experiencing RLS or PLMD symptoms.

At 856, if the patient awoke during delivery of the HF electrostimulation signal and also during experiencing RLS or PLMD symptoms, the parameter adjustment 850 can involve using that information to infer that the previous HF electrostimulation signal parameters were not strong enough to mitigate the RLS or PLMD symptoms. As such, the parameter adjustment 850 can involve facilitating increasing an electrostimulation signal intensity (e.g., increasing a signal amplitude or a signal ramping rate).

At 858, if the patient awoke during delivery of the HF electrostimulation signal but did not awake during RLS or PLMD symptoms, the parameter adjustment 850 can involve using that information to infer that the previous HF electrostimulation signal parameters contributed to the patient awakening. As such, the parameter adjustment 850 can involve facilitating decreasing an electrostimulation signal intensity (e.g., decreasing a signal amplitude or a signal ramping rate).

If the patient did not awake during delivery of the HF electrostimulation signal but did awake during experiencing RLS or PLMD symptoms, the parameter adjustment 850 can involve determining whether a battery of a leg-wearable electrostimulation device (e.g., battery 710 of FIG. 7) became depleted during the delivering electrostimulation during a first sensing period. At 860, if the battery was depleted during the first sensing period, the parameter adjustment 850 can involve tuning a sensitivity of initiation of electrostimulation during a subsequent second sensing period, such as to reduce false positives. Also, the parameter adjustment 850 can involve reducing an electrostimulation duration during the subsequent second sensing period as a power-saving measure. At 862, if the battery was not depleted during the first sensing period, the parameter adjustment 850 can involve determining that the processor circuitry 702 failed to initiate delivery of the electrostimulation in time to prevent or mitigate the determined actual or predicted sleep arousal.

At 864, if the patient did not awake during delivery of the HF electrostimulation signal or during experiencing RLS or PLMD symptoms, the parameter adjustment 850 can involve using that information to infer that the previous HF electrostimulation signal parameters were acceptable and can maintain the previous HF electrostimulation signal.

Returning to FIG. 8A, the processor circuitry 702 can perform signal preprocessing 816 to facilitate processing of received IMU data, such as the motion data 802A (or similarly, motion data from at least one sensing period 810). For example, the signal preprocessing 816 can facilitate filtering 818, e.g., via one or more filters (e.g., high-pass, low-pass, bandpass, notch, etc.) or frequency analysis. The signal preprocessing 816 can also facilitate classifying 820 of motion data 802A to classify a period of time as at least one of “potential leg movement” or “potential increased RLS symptoms”. In an example, the classifying 820 can be performed using a low-complexity (minimal processing capability and low power consumption) algorithm. For example, such low-complexity classification can be based on one or more threshold crossings or based on signal amplitude. For example, the one or more threshold crossings can include threshold crossings from a leg movement signal (e.g., a rectified or preprocessed signal) from a motion sensor. In an example, the filtered sensor data classifying a period of time as at least one of “potential leg movement” or “potential increased RLS symptoms” can define a first subset of IMU data. In an example, the signal preprocessing 816 can facilitate feature selection 822, e.g., of the first subset of IMU data. The feature selection 822 can calculate and save a feature set, which can help reduce memory usage as compared to storing raw data and also provide an input to train a machine learning model. The feature selection 822 can calculate a feature set for each IMU axes, vector magnitude, or a composite of each axis. In an example, the feature set can include statistics that describe the signal amplitude or other characteristics, such as mean, root mean square, percentiles, maximum, minimum, range, maximum to mean, area under the curve, variation, kurtosis, skewness, or correlation coefficients between IMU axes. The feature set can also include a description of starting, ending, maximum, minimum, changes in position, and rate of changes in position of the IMU sensor using the gravitational vector and/or angle of inclination.

In an example, the feature set can also include one or more features derived from the frequency domain of the signal, e.g., obtained using a fast Fourier transform, such as dominant frequency, power at the dominant frequency, total power, ratio of power at the dominant frequency to total power, frequency bandwidth, spectral centroid, and mean or median frequency. Such features can be derived from only the low frequency range where human movement is most likely to occur or alternatively across all available frequencies. The feature set can also include median/zero crossings, Lempel-Ziv Complexity, entropy rate, and the maximum Lyapunov Exponent, or other features describing the time-frequency domain, e.g., wavelet decompositions and wavelet entropy.

In an example, the calculated feature set can be as an input to train a machine learning algorithm, e.g., to predict a range of target HF electrostimulation signal parameters for delivery to the patient that will be subthreshold to the patient waking while mitigating the RLS or PLMD symptom. The machine learning algorithm can be selected from among many different potential supervised or unsupervised machine learning algorithms. Examples of supervised learning algorithms include artificial neural networks, Bayesian networks, instance-based learning, support vector machines, decision trees (e.g., Iterative Dichotomiser 3, C4.5, Classification and Regression Tree (CART), Chi-squared Automatic Interaction Detector (CHAID), and the like), random forests, linear classifiers, quadratic classifiers, k-nearest neighbor, linear regression, logistic regression, and hidden Markov models. Examples of unsupervised learning algorithms include expectation-maximization algorithms, vector quantization, and information bottleneck method.

In an example, a regression model is used and the model is a vector of coefficients corresponding to a learned importance for each of the features of the feature set. In an example, the machine learning algorithm can implement a regression problem (e.g., linear, polynomial, regression trees, kernel density estimation, support vector regression, random forests implementations, or the like).

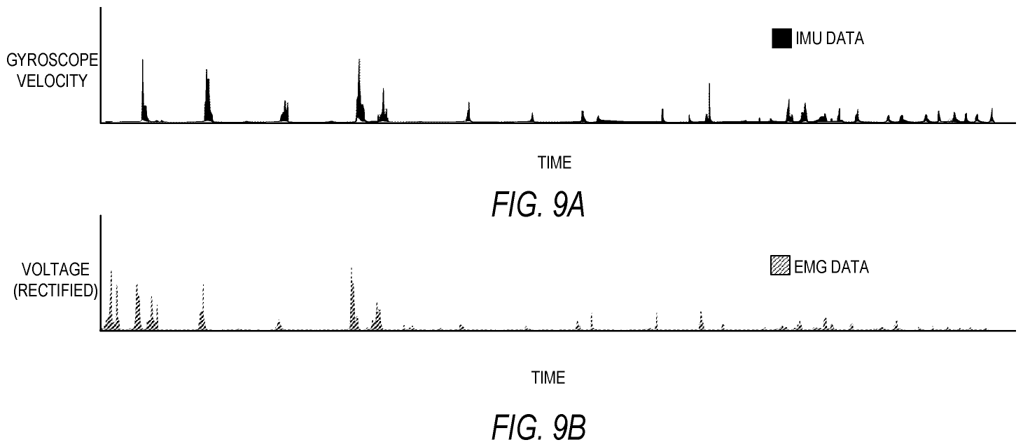

FIG. 9A and FIG. 9B depict IMU data and electromyography (EMG) data, respectively, each over time during a sensing period. In one approach to receiving feedback from electrostimulation delivered to the patient, the RLS treatment system can include at least one EMG electrode for detecting electrical activity in a muscle of a patient limb. An example of such a system is described in US Patent Publication Serial No. US 2021/0100998, which is incorporated by reference herein in its entirety for its teaching of surface electromyography (sEMG) electrodes for personalized patient feedback related to electrostimulation. As depicted in FIG. 9A and FIG. 9B, IMU data including gyroscope velocity over time can resemble rectified voltage data from an EMG electrode. As such, IMU data (as described with respect to FIG. 8A) can be used to approximate electrical activity in a muscle of a patient limb, such as following processing via the signal preprocessing 816 and without needing to receive EMG data. Such approximation can provide advantages over certain EMG approaches in power saving. Also, devices and systems described herein can include or use at least one sEMG electrode for detecting sEMG in a similar fashion to the IMU/motion data. As such, voltage from the sEMG electrode can be rectified, filtered, classified, or subject to feature selection and can be used to help determine a present or future sleep arousal (or to trigger commencing of a present sensing period).

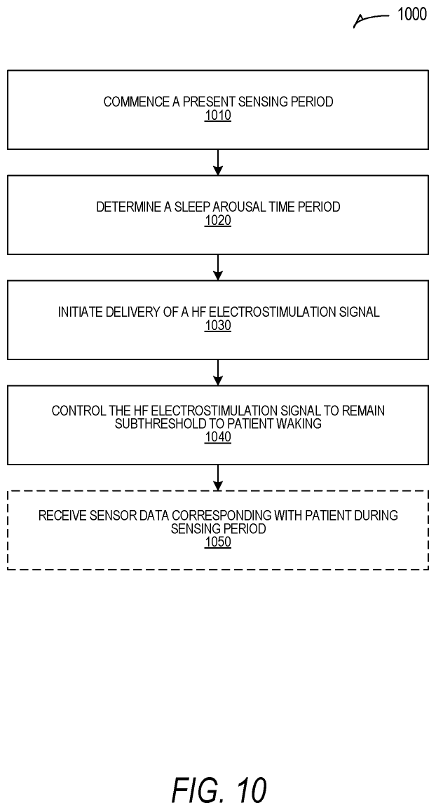

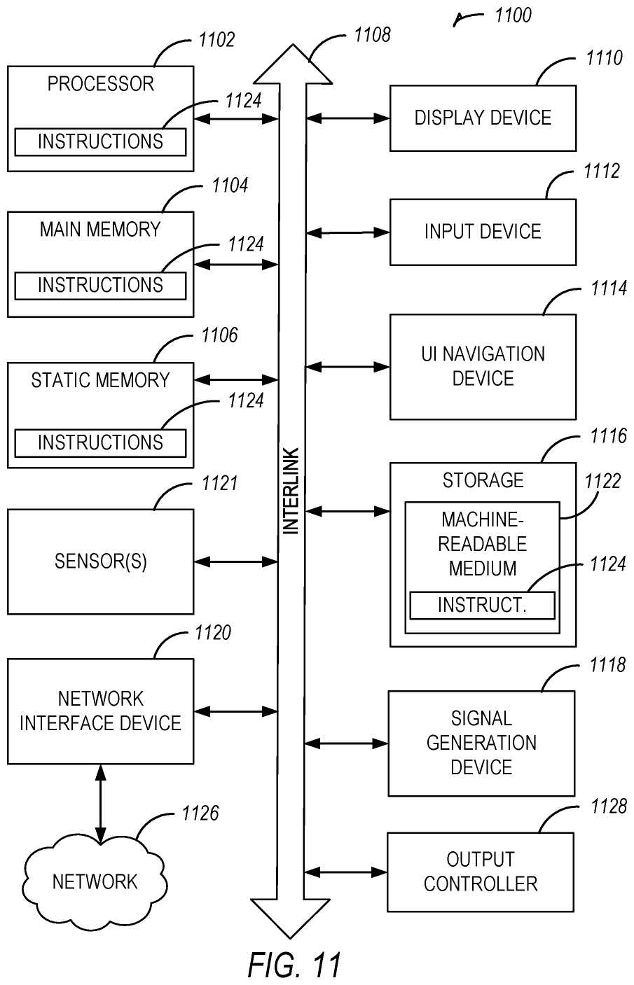

FIG. 10 is a flowchart illustrating a technique 1000 for administering neurostimulation therapy to a patient with Restless Legs Syndrome (RLS) or Periodic Limb Movement Disorder (PLMD). The technique 1000 can be implemented using one or more devices or systems described herein such as the processor of FIG. 11, the processor circuitry 702 of FIG. 7, the device 202A of FIG. 2A, etc.

At 1010, a present sensing period can be commenced, and can optionally be associated with an onset of patient sleep. The present sensing period can be initiated based on various indications of the patient's sleep state, such as changes in the charging status of the device, placement near a patient limb, or direct user input. In an example, the technique can include utilizing motion sensor data, impedance data, or temperature data to determine the onset of sleep.

At 1020, the technique can include determining a sleep arousal time period during the present sensing period. Such a determination can be based on electrostimulation therapy data, which can include movement data or historical data from previous sensing periods. For example, historical data can be used to forecast when the patient is likely to awaken or to adjust therapy parameters based on past patient feedback.

At 1030, upon detecting the sleep arousal time period, delivery of a high-frequency (HF) electrostimulation signal can be initiated or triggered. In an example, the signal's frequency can be maintained between 500 Hz and 15,000 Hz. The technique can include receiving sensor data, such as accelerometer, IMU, or gyroscope data, to help refine the delivery parameters. Additional sensor data, including blood oxygenation or temperature data, can also be used to tailor the therapy.

At 1040, the HF electrostimulation signal is can be controlled to remain subthreshold to the patient waking while maintaining delivery of the HF electrostimulation to treat at least one identified RLS or PLMD symptom. The technique can include establishing or adjusting one or more signal parameters based on IMU data or other sensor inputs. If a previous electrostimulation administration caused the patient to wake, the intensity of the signal can be lowered during a subsequent electrostimulation. The technique can also include using a machine learning model, e.g., trained on IMU data or HF electrostimulation signal frequency data, to predict optimal signal parameters.

Optionally, at 1050, the technique can include receiving sensor data as feedback, the sensor data corresponding with the patient during the sensor period. For example, the technique can include receiving and analyzing IMU data such as to classify leg movements or symptom increases. In an example, a feature set can be calculated based on the received sensor data to inform machine learning models. Additionally, a clinical variable such as user input or a clinical prediction can provide feedback and can be used to influence at least one signal parameter.

FIG. 11 illustrates generally an example of a block diagram of a machine 1100 upon which any one or more of the techniques (e.g., methodologies) discussed herein may perform in accordance with some examples. In alternative embodiments, the machine 1100 may operate as a standalone device or may be connected (e.g., networked) to other machines. In a networked deployment, the machine 1100 may operate in the capacity of a server machine, a client machine, or both in server-client network environments. In an example, the machine 1100 may act as a peer machine in peer-to-peer (P2P) (or other distributed) network environment. The machine 1100 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate on, logic or a number of components, modules, or mechanisms. Modules are tangible entities (e.g., hardware) capable of performing specified operations when operating. A module includes hardware. In an example, the hardware may be specifically configured to carry out a specific operation (e.g., hardwired). In an example, the hardware may include configurable execution units (e.g., transistors, circuits, etc.) and a computer readable medium containing instructions, where the instructions configure the execution units to carry out a specific operation when in operation. The configuring may occur under the direction of the executions units or a loading mechanism. Accordingly, the execution units are communicatively coupled to the computer readable medium when the device is operating. In this example, the execution units may be a member of more than one module. For example, under operation, the execution units may be configured by a first set of instructions to implement a first module at one point in time and reconfigured by a second set of instructions to implement a second module.

Machine (e.g., computer system) 1100 may include a hardware processor 1102 (e.g., a central processing unit (CPU), a graphics processing unit (GPU), a hardware processor core, or any combination thereof), a main memory 1104 and a static memory 1106, some or all of which may communicate with each other via an interlink (e.g., bus) 1108. The machine 1100 may further include a display unit 1110, an alphanumeric input device 1112 (e.g., a keyboard), and a user interface (UI) navigation device 1114 (e.g., a mouse). In an example, the display unit 1110, alphanumeric input device 1112 and UI navigation device 1114 may be a touch screen display. The machine 1100 may additionally include a storage device (e.g., drive unit) 1116, a signal generation device 1118 (e.g., a speaker), a network interface device 1120, and one or more sensors 1121, such as a global positioning system (GPS) sensor, compass, accelerometer, or other sensor. The machine 1100 may include an output controller 1128, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 1116 may include a machine readable medium 1122 that is non-transitory on which is stored one or more sets of data structures or instructions 1124 (e.g., software) embodying or utilized by any one or more of the techniques or functions described herein. The instructions 1124 may also reside, completely or at least partially, within the main memory 1104, within static memory 1106, or within the hardware processor 1102 during execution thereof by the machine 1100. In an example, one or any combination of the hardware processor 1102, the main memory 1104, the static memory 1106, or the storage device 1116 may constitute machine readable media.

While the machine readable medium 1122 is illustrated as a single medium, the term “machine readable medium” may include a single medium or multiple media (e.g., a centralized or distributed database, or associated caches and servers) configured to store the one or more instructions 1124.

The term “machine readable medium” may include any medium that is capable of storing, encoding, or carrying instructions for execution by the machine 1100 and that cause the machine 1100 to perform any one or more of the techniques of the present disclosure, or that is capable of storing, encoding or carrying data structures used by or associated with such instructions. Non-limiting machine-readable medium examples may include solid-state memories, and optical and magnetic media. Specific examples of machine-readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 1124 may further be transmitted or received over a communications network 1126 using a transmission medium via the network interface device 1120 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 1102.11 family of standards known as Wi-Fi®, IEEE 1102.16 family of standards known as WiMax®), IEEE 1102.15.4 family of standards, peer-to-peer (P2P) networks, among others. In an example, the network interface device 1120 may include one or more physical jacks (e.g., Ethernet, coaxial, or phone jacks) or one or more antennas to connect to the communications network 1126. In an example, the network interface device 1120 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 1100, and includes digital or analog communications signals or other intangible medium to facilitate communication of such software.

The above description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A method for performing neurostimulation therapy for a Restless Legs Syndrome (RLS) or Periodic Limb Movement Disorder (PLMD) patient, the method comprising:
commencing a present sensing period;
determining a sleep arousal time period, during the present sensing period, associated with actual or predicted patient transition from asleep to awake;
initiating, during the sleep arousal time period during the present sensing period, delivery of a high-frequency (HF) electrostimulation signal to a target location of the patient at a frequency between 500 Hz and 15,000 Hz, the HF electrostimulation signal configured to mitigate an RLS or PLMD symptom; and
controlling the HF electrostimulation signal toward parameters subthreshold to the patient waking while maintaining the delivery of the HF electrostimulation signal for mitigating the RLS or PLMD symptom.

2. The method of claim 1, wherein the present sensing period is based on an indication of present or expected sleep onset of the patient, indication of sleep onset including at least one of:
a change in charging status of an electrostimulation device associated with the HF electrostimulation signal;
an indication of the electrostimulation device being placed proximate to a patient limb;
a received user input;
an indication of a present time of day; or
a time of day of a previous user-activated electrostimulation delivery.

3. The method of claim 2, comprising determining the indication of present or expected sleep onset of the patient based on at least one of:
motion sensor data;
impedance data of the electrostimulation device; or
temperature data of the electrostimulation device.

4. The method of claim 1, wherein the determining the sleep arousal time period is based at least in part on electrostimulation therapy data that includes movement data detected from the patient during the present sensing period.

5. The method of claim 1, wherein the determining the sleep arousal time period is based at least in part on electrostimulation therapy data that includes historical data from at least one previous sensing period associated with the same patient and occurring before the present sensing period.

6. The method of claim 5, wherein:
the controlling the HF electrostimulation signal toward parameters subthreshold to the patient waking while maintaining the delivery of the HF electrostimulation signal for mitigating the RLS or PLMD symptom includes establishing or adjusting at least one parameter of the HF electrostimulation signal, during the present sensing period, based on the historical data from the at least one previous sensing period; and
the historical data includes:
waveform data corresponding with an electrostimulation administration during the at least one previous sensing period; and
patient feedback data detected during the at least one previous sensing period and corresponding with the waveform data.

7. The method of claim 6, comprising establishing a lower intensity of the HF electrostimulation signal, as compared to that of the individual previous sensing period, based on historical data including patient feedback data and corresponding waveform data indicating that the electrostimulation administration caused the patient to wake during the at least one previous sensing period.

8. The method of claim 5, wherein the determining the sleep arousal time period includes forecasting when a patient is likely to awaken during the present sensing period based on the historical data from the at least one previous sensing period.

9. The method of claim 5, wherein the electrostimulation therapy data includes historical data corresponding with respective sensing periods corresponding with a plurality of different RLS or PLMD patients.

10. The method of claim 1, comprising:
receiving sensor data corresponding with the patient during the present sensing period; and
wherein controlling the HF electrostimulation signal toward parameters subthreshold to the patient waking includes establishing or adjusting at least one parameter of the HF electrostimulation signal based on the received sensor data.

11. The method of claim 10, wherein the sensor data includes at least one of inertial measurement unit (IMU) data, accelerometer data, or gyroscope data.

12. The method of claim 10, wherein the sensor data includes at least one of blood oxygenation data, a pulse oximeter data, optical heart sensor data, a photoplethysmography (PPG) sensor data, audio physiological sensor data, audio environment sensor data, temperature sensor data, heat flux sensor data, or circadian/pattern detector data.

13. The method of claim 1, wherein commencing the present sensing period is based on an indication that an electrostimulation device corresponding with the HF electrostimulation signal is being worn by the patient.

14. The method of claim 1, wherein controlling the HF electrostimulation signal toward parameters subthreshold to the patient waking includes establishing or adjusting at least one signal parameter of the HF electrostimulation signal based on inertial measurement unit (IMU) data received during the sensing period and from a sensor circuit of an electrostimulation device associated with the HF electrostimulation signal.

15. The method of claim 14, comprising processing the IMU data to classify a first subset of the IMU data subset indicating at least one of potential leg movement or potential increased symptoms.

16. The method of claim 15, comprising calculating at least one feature set of the first subset of the IMU data, the first feature set including an indication of a change in position of an IMU sensor using a gravitational vector or angle of inclination.

17. The method of claim 16, comprising using the calculated at least one feature set as an input to train a machine learning model to predict a range of target HF electrostimulation signal parameters for delivery to the patient that will be subthreshold to the patient waking while mitigating the RLS or PLMD symptom.

18. The method of claim 15, comprising calculating at least one feature set of HF electrostimulation signal frequency data, corresponding the first subset of IMU data, using a fast Fourier transform (FFT).

19. The method of claim 18, comprising using the calculated at least one feature set as an input to train a machine learning algorithm to predict a range of target HF electrostimulation signal parameters for delivery to the patient that will be subthreshold to the patient waking while mitigating the RLS or PLMD symptom.

20. The method of claim 1, wherein controlling the HF electrostimulation signal includes establishing or adjusting at least one signal parameter of the HF electrostimulation signal based on at least one clinical variable corresponding with the patient, the clinical variable including at least one of a user input, a clinical prediction of a time period the patient is likely to wake, or a specified patient symptom.

21. A method for performing neurostimulation therapy for a Restless Legs Syndrome (RLS) or Periodic Limb Movement Disorder (PLMD) patient, the method comprising:
commencing a present sensing period;
determining a sleep arousal time period, during the present sensing period, associated with actual or predicted patient transition from sleep to wake; and
initiating, during the sleep arousal time period during the present sensing period, delivery of a high-frequency (HF) electrostimulation signal to a target location of the patient at a frequency between 500 Hz and 15,000 Hz, the HF electrostimulation signal configured to mitigate an RLS or PLMD symptom.

22. A computing device for facilitating neurostimulation therapy for a Restless Legs Syndrome (RLS) or Periodic Limb Movement Disorder (PLMD) patient, the computing device including a processor and a memory device, the memory device including instructions that, when executed by the processor, cause the computing device to:
commence a present sensing period;
determine a sleep arousal time period, during the present sensing period, associated with actual or predicted patient transition from sleep to wake; and
initiate, during the sleep arousal time period during the present sensing period, delivery of a high-frequency (HF) electrostimulation signal to a target location of the patient at a frequency between 500 Hz and 15,000 Hz, the HF electrostimulation signal configured to mitigate an RLS or PLMD symptom; and
control the HF electrostimulation signal toward parameters subthreshold the patient waking while maintaining the delivery of the HF electrostimulation signal for mitigating the RLS or PLMD symptom.
